(19) United States
(12) Patent Application Publication
(10) Pub. No.: US 2007/0185012 A1
(43) Pub. Date: Aug. 9, 2007

(54) Glycopeptides for the treatment of als and other metabolic and autoimmune disorders

(76) Inventors: V.J. Rajadhyaksha, Mission Viejo, CA (US); Thomas P. Lahey, Laguna Niguel, CA (US)

Correspondence Address: Robert D Buyan;Stout Uxa Buyan & Mullins, Suite 300, 4 Ventura, Irvine, CA, 92618, US

(21) Appl. No.: 10/564,324

(22) PCT Filed: Jul. 9, 2004
(86) PCT No.: PCT/US04/22134

§ 371 Date: Jun. 30, 2006

Related U.S. Application Data
(60) Provisional application No. 60/486,350, filed on Jul. 10, 2003.

(51) Int. Cl. A61K 38/14

(52) U.S. Cl. 514/8

(57) ABSTRACT

New compositions and methods for treating patients suffering from Amyotrophic Lateral Sclerosis (ALS) and other metabolic and autoimmune disorders, which include glycopeptides such as N-acetyl-D-glucosaminyl(β1-4)-N-Acetyl-muramyl-L-alanyl-D-isoglutamine (GMDP) and peptide analog-L-alanyl-D-glutamic acid (GMDP-A) of at least 98% purity administered either alone, or in combination with a flavone such as luteolin and/or an isoflavone such as genistein, optionally in combination with a flavonol glycoside such as isoquercitrin or rutin. The high purity glycopeptides have a decreased amount immunogenic impurities and demonstrate a synergistic effect when combined with luteolin and/or genistein in presence of isoquercitrin.

### RELATED APPLICATION

This patent application claim priority to U.S. Provisional Application No. 60/486,350 filed on Jul. 10, 2003, the entirety of which is expressly incorporated herein by reference.

### FIELD OF THE INVENTION

This invention relates to new compositions and methods for treating patients suffering from metabolic and autoimmune disorders and more particular compositions and methods for treating Amyotrophic Lateral Sclerosis (ALS).

### BACKGROUND OF THE INVENTION

Metabolic disorders, such as Amyotrophic Lateral Sclerosis (ALS), whose cause remains unknown to date, is a neurodegenerative disorder characterized by the inexorable degeneration of upper motoneurons in the motor cortex and lower motoneurons in the brainstem and spinal cord. As with other degenerative disorders of the CNS, such as Parkinson's disease and Alzheimer's disease, familial and sporadic forms of ALS are observed. The prognosis of ALS is very severe, being linked mainly to the involvement of respiratory and bulbar muscles. No biologic markers of ALS have yet been discovered and daily life scale and functional muscle tests are the best way to follow the natural evolution of patients or the clinical status of patients involved in clinical trials.

The recent advances in the understanding of neuronal degeneration in ALS and the identification of a gene mutation in familial ALS have led to the discovery of new therapeutic agents such as Riluzole and perhaps insulin-like growth factor I (IGF-I), which have shown efficacy in slowing the evolution of the disease.

There are currently four main hypotheses about the cause of ALS: excitotoxicity linked to glutamate receptor overactivation; mutation of the superoxide dismutase gene; production of autoantibodies to voltage-dependant calcium channels resulting in disordered function; neurofilament accumulation. Furthermore, the activity of protein kinase C (PKC), a Ca⁺⁺ phospholipid dependent enzyme, is also substantially increased in tissue from ALS patients, suggesting that alterations in intracellular free Ca⁺⁺ may be central to many of the diverse pathogenic mechanisms potentially responsible for ALS. Increased PKC activity may influence neuronal viability and the pathogenic process in ALS by modifying the phosphorylation of voltage-dependent Ca⁺⁺ channels, neurotransmitter receptors and structural proteins (Krieger et al 1996. Trends in Pharmacological Sciences: 17, 114). The motoneuron degeneration characteristic of ALS could be caused by any one or a combination of these mechanisms. Future therapeutic approaches, based on these mechanisms, should be combination therapies so that different levels of the degenerative process are targeted. Protection against excitotoxicity could be achieved with a combination of pharmacological agents having neuroprotective activity, such as antiglutamate agents (e.g., riluzole), N-methyl-D-aspartate (NMDA) and non-NMDA antagonists, free-radical scavengers, calcium-channel blockers, and neurotrophic factors. To date, NMDA antagonists have not shown efficacy in modifying the clinical evolution of ALS. AMPA/kainate antagonists are still under development. Drugs that reduce glutamate release from presynaptic terminals have been tested but to date only riluzole has been proven to have some efficacy. (Hugon, 1996. Neurology 47, S251-254).

Reactive oxygen species (ROS) and free radicals induce membrane damage, oxidation of DNA bases, breaks in DNA strands, chromosomal aberrations and alterations in protein structure. Therefore, oxidative stress is closely associated with aging, atherosclerosis and cancer.Oxidative stress clearly plays a role in the pathogenesis of ALS, thus indicating a relationship between excitotoxicity and the production of free radicals. Living cells are normally protected by antioxidant enzymes, such as SOD, and free radical scavengers including tocopherols and glutathione. The latter is depleted in cells under oxidative stress due to elevated levels of γ-glutamyl transpeptidase (GGTP), which hydrolyzes glutathione into its components. Inhibitor of this enzyme would alleviate the problem and help maintain the levels of this antioxidant (Slesarev, wide infra, U.S. Patent Application Publication 2001/0034325). However, in a randomized, doubleblind, controlled trial N-acetylcysteine, a free-radical scavengers, showed only partial efficacy in ALS patients (Louverse et al 1995. Arch. Neurol. 52, 559-564).

Glycopeptides constitute a broad class of organic compounds comprising substances including the sugar part and the peptide part and incorporate an unusual carboxyl-containing aminosugar generally referred to as N-acetylmuramic acid (MurNAc), having the 2-deoxy-β-D-glucopyranose structure with an acetamido group in the 2-position and a (D-I′-carboxyethyl) group on the oxygen in the 3-position. Glycopeptides of bacterial cell walls comprise gigantic polymeric molecules composed of alternating units of a disaccharide GlcNAc(β I,4)MurNAc [N-acetyl-glucosaminyl (β I-4) N-acetylmuramic acid] with peptides of a similar structure bonded through the carboxyl group of muramic acid. The peptide bonds between basic peptide chains of these glycopeptides contribute to rigidity of the cell wall structure of bacteria. By means of a specific enzymatic hydrolysis it is possible to break-up certain bonds in a polymeric molecule of glycopeptides of cell walls of bacteria and obtain glycopeptide fragments of a various length and various structure (Ghuysen J. M., Bact. Rev. 32, U.S. Pat. No. 4,425,1968, and Schliefer K. H., Kandler O., Bact. Rev. 36, U.S. Pat. No. 4,407,1972).

U.S. Pat. No. 4,395,399, issued to Ovchinnikov et. al. describes several glycopeptides and their method of preparation and is hereby incorporated by reference in its entirety. The semi-synthetic pathway comprises condensation of an aminosugar component with a bloclced amino acid or peptide component, followed by deblocking of the protecting groups. The aminosugar component is obtained from enzymatic hydrolysis of bacterial cell wall biomass with lysozyme, followed by ion-exchange chromatography. GMDP was also isolated during analysis of the anti-tumor drug, blastolysine, which is a lysozyme cell wall hydrolysate of Lactobacillus Bulgaricus. (U.S. Pat. No. 4,395,399) GMDP has been extensively studied in animals, demonstrating adjuvant activity, antitumor activity, low pyrogenicity, and hypnogenic effect. (Andronova T., et al., Sov. Med. Rev. lmmunol.; 4:1-63(1991)). Although the glycopeptides of cell walls of bacteria provide rigidity and protection to the cell walls it has been found that numerous glycopeptides recovered from bacterial cell walls are strong adjuvants (Ellous F., Adam A., Ciorbaru R., Lederer E., Biochem. Biophys. Res. Comm. 59, No. 4, 1317 (1974).

Ledger, Int. Pat. Appl. 1996, WO 96/01645, which is hereby incorporated by reference in its entirety, describes glycopeptide compounds, particularly N-acetyl-D-glucosaminyl-(β1,4)-N-acetylmuramyl-L.alanyl-D-isoglutamine (GMDP), useful in the treatment or prophylaxis of inflammatory dermatological conditions such as psoriasis and in the treatment or prophylaxis of immune-related diseases of the skin and mucous membranes. It also claims simple MDP analogs, which are not useful for therapeutic applications of this invention. MDP, the minimum bacterial cell wall glycopeptide component and lipopolysaccharide (LPS) induce inflammatory responses similar to those induced by bacterial endotoxins and support their pathogenic role in bacterial infections. MDP was associated with glutamate release, decreased GGTP levels and renal cell apoptosis in rabbits. MDP has been shown to induce edema, fever, sleep, loss of appetite, arthritis, uveitis and epithelial cell toxicity (Langford et al 2002. Mol. Cell. Biochem. 236, 63-73).

U.S. Pat. No. 6,281,191 issued to Slesarev et. al. describes new compositions and methods for treating hepatitis-C, AIDS and aberrant apoptosis, which include GMDP of at least 98% purity alone or in combination with N-acetyl glucosamine (NAG) and is hereby incorporated by reference in its entirety. GMDP and other muramyl dipeptides have shown inhibitory effects on lipopolysaccharide (LPS) induced TNF-α, which results in preventing the toxic action of LPS during septic shock. (Adeleye T. A., et al., APMIS.; 102:145-152(1994)). However, Slesarev et al (wide supra) have shown that decreasing or eliminating LPS and polysacchrides levels increases the apoptosis regulating properties of GMDP and have demonstrated that the purity level of the GMDP affects its ability to protect cells and modulate apoptosis by concurrent inhibition of both TNF-α and Fas antigen mediated cytotoxicity without extensive detrimental immunostimulation. According to Slesarev et al, N-acetyl-D-glucosaminyl-(β1,4)-N-Acetyl-muramyl-L-alanyl-D-isoglutamine (GMDP) is used to modulate Fas mediated apoptosis and stimulate TNF-α production and selectively inhibit its p55(TNFR1) receptor.

U.S. Patent Application Publication 2001/0034325, filed by Slesarev and hereby incorporated by reference in its entirety, describes preparation of glycoproteins and glycopeptides from Lactobacillus Reuteri and compositions with and without NAG for lowering γ-glutamyl transpeptidase, which restores glutathione levels, which appears to be connected with several cardiovascular, neurological and oncological diseases. GGTP inhibition leads to the preservation of extracellular glutathione which is a powerful antioxidant with remarkable detoxification properties. The low molecular weight glycopeptides, MDP (492 D) and impure GMDP (695 D), are mainly responsible for immunogenic effects. They are potent stimulators of TNF-α production, which may be detrimental in patients with autoimmune conditions such as rheumatoid arthritis. Excessive level TNF-α production could be extremely dangerous for patients with ARDS, stroke, and ischemic heart disease, who already have high preexisting production of TNF-α. Moreover, combination of MDP and TNF-α can cause proinflammatory effects, thus exaggerating chronic viral and bacterial infection. The glycoproteins having molecular weight higher than 800 D and less than 30000 D obtained by lysozyme hydrolysis of gram positive bacteria exhibit newly discovered antioxidant and detoxification effects. In parallel, undesirable proinflammatory and immunogenic properties were avoided by eliminating low molecular weight glycopeptides, MDP and GMDP. It provides exceptional safety and improved tolerance in people with autoimmune conditions. In addition, achieved GGTP inhibition leads to the repletion of glutathione, well-known TNF-α inhibitor. These antioxidants and free radical scavenging glycopeptide inhibitors of GGTP are of natural origin (originating from lactic acid bacteria, which are used in the production of yogurt and fermented milk food and drinks).

All of the glycoprotein compositions, obtained from various Lactobacillus (L.) strains, including L. acidophilus, L. casei, L. bifermentans, L. reuteri, L. alimentarius, L. helveticus, L. brevis, L. collinoides, L. coryneformis, L. crispatus, L. curuvatus, L. delbrueckii, L. jensenii, L. lactis, L. salivarus, L. murinus, L. Bulgaricus, L. Plantarum, etc., are natural products and can be obtained with more than 98% purity when processed properly.

Several groups have reported total synthesis of glycopeptides (See for example, Ledvina et al, 1998. Coll. Czech. Chem. Comm. 63, 577-589 and references cited therein; Kiso et al, 1982. Carbohydrate Res. 104, 253-269; Blaszczak et al, PCT Int. Appl. 2001 WO 0179267 and WO 0179268; Vosika & Ma, PCT Int. Appl. 1997 WO 9712894 and Bezonikova, PCT Int. Appl. 1997 WO 9710259).

The cellular and molecular basis for activity of GMDP, despite its interest as a therapeutic compound, are not fully understood. GMDP can modulate vascular endothelial cells without induction of angiogenesis, a highly complex process depending on promoters and inhibitors such as growth factors, cytokines, adhesion molecules and the extracellular matrix. Endothelial cells play an important role in tissue homeostasis, immune modulation and signal transduction and their dysfunction has been implicated in the pathology of a variety of vascular disorders including chronic inflammation. It has been suggested that GMDP probably suppresses the secretion of angiogenic factors such as TNF-α and IL-Iα, but the existence of alternative mechanisms is also possible. GMDP, being non-angiogenic, may have potential as a therapeutic agent for treatment of tumor growth and metastases, and inflammatory diseases, such as psoriasis and rheumatoid arthritis potentiated by angiogenesis (Li et. al. 1997 Inflamm. Res. 46, 348).

Physiological cell death mostly proceeds by apoptosis, a process which may be Fas-induced or TNF-α triggered is either impaired or overactive contributing to a number of disease conditions, such as hepatitis-C, autoimmune disorders, diabetes, acute pancreatitis and numerous other disorders. Normalizing or modulating the apoptotic process to carry out its important biological processes would lead to therapeutic treatment for many of these degenerative diseases and disorders. Fas antigen and TNF-α receptor blocking mechanism would allow for the design of an efficient treatment for apoptosis associated with the above-mentioned disorders. In this respect, muramyl peptides are regarded as most promising stimulators. D-peptidoglycans namely N-acetyl-D-glucosaminyl-(β-1,4)-N-acetylmuramyl-L-alanyl-D-isoglutamine (GMDP) have been proposed as the cytotoxic agents capable of eliminating cancer cells and/or virus infected cells. (Ovchinnikov, et al. U.S. Pat. No. 4,395,399).

Given the shortcomings associated with the currently available modes of therapy for metabolic and autoimmune disorders, particularly ALS, there remains a need for the development of new therapeutics and particularly nutraceuticals that are effective and with minimal side effects. To our knowledge, treatment of ALS with glycopeptides having molecular weights higher than 800 D has not been reported. We have now surprisingly found that a composition comprising one or more glycopeptides, either alone or in combination with free radical scavengers is therapeutically safe and effective for the treatment of ALS and other metabolic and autoimmune disorders. The combination of a glycopeptide and a free-radical scavenger certainly could provide strong neuroprotective activity by acting at two different targets upstream and downstream in the degenerative process. Such free radical scavengers to combat oxidative stress include, for example, glutathione and its derivatives, lipoic acid, Vitamins C and E, N-acetyl cysteine, thiazolidin-2-one-4-carboxylic acid and flavones, isoflavones and flavonol glycosides, described in our copending U.S. application Ser. No. 10/236,86, entitled Inhibition By 3-Deoxyflavonoids of T-Lymphocyte Activation and Therapies Related Thereto, filed on Sep. 6, 2002, which is hereby incorporated by reference in its entirety. Furthermore, we have now discovered that the therapeutic effect of glycopeptides with administration of a flavone and/or an isoflavone, optionally in combination with a flavonol glycoside, which enhances the efficacy of the flavone or Isoflavone, is synergistic rather than additive. Additionally, the compositions may further include a glutamate, NMDA or AMPA/kainate antagonist and a COX inhibitor.

In determining the efficacy of a drug and the effectiveness of the use of a drug to treat a disease, drug absorption is a critical concern. Drug absorption refers to the process of drug movement from the site of administration toward the systemic circulation. Typically, therapeutic drugs are administered parenterally or enterally. Of course, parenteral administration is the administration of the drug intravenously directly into the blood stream and although this mode of administration provides a method for eliminating a number of the variables that are present with oral administration, parenteral administration is not a preferable route of choice for many therapeutic compounds. Enteral refers to the administration of the drug into the gastrointestinal tract.

Oral administration of drugs is by far the most common method. When administered orally, drug absorption usually occurs by passive diffusion across the membranes of the epithelial cells within the gastrointestinal tract. Absorption after oral administration is confounded by numerous factors. These factors include differences down the alimentary canal in: the luminal pH; surface area per luminal volume; perfusion of tissue, bile, and mucus flow; and the epithelial membranes. A further issue effecting the absorption of orally administered drugs is the form of the drug. Most orally administered drugs are in the form of tablets or capsules. This is primarily for convenience, economy, stability, and patient acceptance. Accordingly, these capsules or tablets must disintegrate or dissolve before absorption can occur. There are a variety of factors capable of varying or retarding disintegration of solid dosage forms. Further, there are a variety of factors that effect the dissolution rate and therefore determine the availability of the drug for absorption.

Not only is drug absorption an issue in drug delivery but also the bioavailability of the drug is also critical. Bioavailability is defined as the rate at which and the extent to which the active moiety (drug or metabolite) enters the general circulation, thereby gaining access to the site of action. Bioavailability depends upon a number of factors, including how a drug product is designed and manufactured, its physicochemical properties, and factors that relate to the physiology and pathology of the patient. An orally administered drug must pass through the intestinal mucosa and the liver, both of which are abundant in enzymes that will rapidly and effectively metabolize the drug in many ways, thereby reducing the plasma concentration of the drug and its effectiveness to a very short period of time following the oral administration. A large number of drugs show low bioavailability owing to an extensive first pass metabolism. Bioavailability considerations are most often encountered for orally administered drugs and can have profound clinical significance.

This metabolic breakdown of the active drug may be circumvented by mucosal administration of the drug. Examples of such mucosa include, for example, buccal or sublingual, nasal (Chien et al., 1987. “Intranasal Drug Delivery for Systemic Medications,” CRC Critical Reviews in Therapeutic Drug Carrier Systems, 4:67-194), vaginal, rectal, dermal and pulmonary (Byron et al., 1994. Journal of Aerosol Medicine, 7, 49-75). Drugs administered by these routes avoid gut-wall and hepatic metabolism, thereby producing increased bioavailability as compared to oral administration. Our copending U.S. Provisional Patent Application No. 60/407,125 entitled uParenteral Administration of 3-Dexoyflavinoids to Avoid First Pass Metabolism” filed Aug. 30, 2002, describes the advantages of mucosal, particularly buccal, administration of flavones and it is herein incorporated by reference in its entirety and is hereby incorporated by reference in its entirety.

Nasal drug administration serves as an alternative route of drug administration. It has been shown that most drugs administered nasally produce plasma levels similar to those following intravenous administration (Hussain, et al., 1980. J. Pharm. Sci., 69, 1240; Bawarshi-Nassar et al., 1989. J. Pharm. Pharmacol 41, 214; Hussain, et al., 1979. J. Pharm. Sci. 68, 1196). The nasal delivery route is a very useful method of drug administration, which frequently improves drug bioavailability by direct absorption into the circulation avoiding hepatic first-pass metabolism and destruction in the gastrointestinal tract observed following oral delivery of drugs (Chien, et al., Marcel Dekker, New York, 1989).

We have now surprisingly found that the bioavailability of the glycopeptides, for example, DMGP or DMGP-A, of the present inventions is improved by administering the compound via the nasal route. We also found that intranasal delivery of the glycopeptide enabled us to reduce the dose required for its beneficial effect and improved drug bioavailability by direct absorption into the circulation as compared to low bioavailability and significantly lower plasma concentrations of orally administered glycopeptide GMDP (Lyons et al 2000. Int. J. Pharm. 199, 17-28). Therefore, small doses of GMDP can be administered which will results in fewer side effects, and the drug will be more tolerable and more effective in treating patients suffering from ALS or other metabolic and autoimmune disorders. Furthermore, compliance with nasal delivery is expected to be higher in ALS patients due to difficulty in swallowing orally administered medications.

### SUMMARY OF THE INVENTION

The present invention provides new compositions and methods for treating patients suffering from autoimmune and/or metabolic disorders, including but not limited to Amyotrophic Lateral Sclerosis (ALS), Multiple Sclerosis (MS), Type I Diabetes, Rheumatoid Arthritis, Psoriasis, etc. A therapeutically effective amount of a composition comprising one or more glycopeptides of Formula I is administered, either alone or in combination with free radical scavengers as therapeutically safe and effective for the treatment of ALS and other metabolic and autoimmune disorders. The compositions may further include a glutamate, NMDA or AMPA/kainate antagonist and a COX inhibitor. 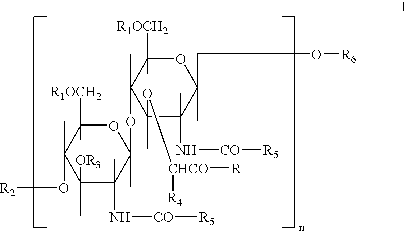
wherein:
R₁, R₂ and R₃ each represents a hydrogen atom or a C₁-C₂₂ acyl group;
R₄ represents a hydrogen atom or a C₁-C₆ alkyl group;
R₅ represents a C₁-C₂₁ alkyl group or a C₆ or C₁₀ aryl group;
R₆ represents a hydrogen atom; and
R represents the residue of an amino acid or a linear peptide of up to from 2 to 6 amino acid residues. Furthermore, at least one of the residues may be optionally substituted with a lipophilic group through an ester or amide bond; and n is 1 and 2.

Further, in accordance with this invention, the compound of the above-set-forth general formula may be administered in combination (e.g., before, simultaneously with or after) a flavone or flavone derivative (e.g., a flavinoid or flavinol), isoflavone or isoflavone derivative, or a prodrug or a congener thereof. Moreover, an advantage of the present invention is to provide an improved method for delivery of the glycopeptides and flavone derivatives to increase their bioavailability. The composition permits administration of glycopeptides and flavonoids through the mucosal membrane. The preferred mucosal membranes are nasal, rectal, vaginal, dermal, buccal and sublingual. The most preferred mode of administration for glycopeptides is the nasal route, whereas the most preferred mode of administration for flavonoids is the buccal/sublingual route as previously described in co-pending U.S. Provisional Patent Application No. 60/407,125 entitled “Parenteral Administration of 3-Dexoyflavinoids to Avoid First Pass Metabolism” filed Aug. 30, 2002 referred to and incorporated hereabove.

Still further, an advantage of the present invention is to provide a method of delivering nutraceutical and therapeutic agents to an individual that provides for increased absorption and bioavailability as compared to medicaments that are designed to be absorbed in the GI tract.

Further, an advantage of the present invention is to provide a method of administering a nutraceutical and therapeutic agents to an individual at a lower level than is typically administered orally while still achieving the same effect.

Furthermore, an advantage of the present invention is to provide a method for administering nutraceutical and therapeutic agents to an individual that heretofore were administered orally.

Moreover, an advantage of the present invention is to provide an improved method for delivery. The composition permits administration of glycopeptides, particularly GMDP and GMDP-A, through nasal, buccal or sublingual mucosa, for attaining sustained blood levels of the active agent.

Still further, an advantage of the present invention is to provide a method that permits simultaneous, separate or sequential administration of flavones (e.g., flavonoids), for example luteolin or its derivatives, through the membranes of the mouth, buccally or sublingually, for attaining sustained blood levels of this active agent.

A method of providing therapy using the pharmaceutical composition of the present invention comprises the application of a dosage form according to this invention to the nasal mucosa, buccal pouch or under the tongue of a subject, such as a human.

Accordingly, a major object of the present invention is to provide a composition and method for the safe, convenient and effective way of administering the glycopeptides to a patient in need of such treatment. The method comprises intranasal administration of an effective amount of a glycopeptide, for example, GMDP or GMDP-A, for the treatment of ALS or other metabolic and autoimmune disorders.

In addition, nasal GMDP administration Is easy and convenient in ALS patients, where swallowing of oral dosage forms is painful and difficult. Furthermore, in many situations it has already been shown that the onset and extent of drug delivery after intranasal administration is comparable to the same drug and dose being given intravenously. Therefore, intranasal delivery of GMDP for treatment of ALS or other metabolic and autoimmune disorders could be used in those situations where a rapid or intermittent drug effect is desired.

In certain embodiments, the invention is directed to a method of providing glycopeptide therapy to a patient in need thereof comprising intranasally administering an effective amount of a glycopeptide or a pharmaceutically acceptable derivative thereof to said patient and compositions thereof. Preferably, the glycopeptide is administered with a pharmaceutically acceptable carrier which can be in the form of, e.g. a solution, suspension, gel, ointment, lotion, semi-solid, vaporizable carrier, a powder and combination thereof. In certain embodiments, the carrier can provide a sustained release of the drug.

### DETAILED DESCRIPTION OF THE INVENTION

The present invention describes new compositions and methods for treating patients suffering from metabolic and autoimmune disorders and more particular compositions and methods for treating Amyotrophic Lateral Sclerosis (ALS). More particularly, it describes a composition comprising one or more glycopeptides of Formula I, either alone or in combination with free radical scavengers as therapeutically safe and effective for the treatment of ALS and other metabolic and autoimmune disorders. The compositions may further include a glutamate, NMDA or AMPA/kainate antagonist and a COX inhibitor.

Many therapeutically useful glycopeptide compounds of this invention are represented by general formula I: 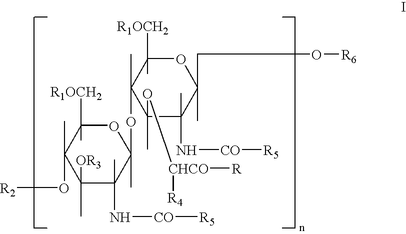
wherein:
R₁, R₂ and R₃ each represents a hydrogen atom or a C₁-C₂₂acyl group;
R₄ represents a hydrogen atom or a C₁-C₆ alkyl group;
R₅ represents a C₁-C₂₁ alkyl group or a C₆ or C,₀ aryl group;
R₆ represents a hydrogen atom; and
R represents the residue of an amino acid or a linear peptide of up to from 2 to 6 amino acid residues. Furthermore, at least one of the residues may be optionally substituted with a lipophilic group through an ester or amide bond; and n is 1 and 2.

Preferred acyl groups for R₁, R₂ and R₃ are C₁-C₅ acyl groups (not including the carbonyl moiety) such as acetyl. Preferred alkyl groups for R₄ are C₁-C₄ alkyl groups such as methyl and ethyl. Preferred alkyl groups for R₅ are C₁-C₆ alkyl groups, particularly C₁-C₄ alkyl groups such as methyl or ethyl; phenyl is a preferred aryl group.

R preferably represents an amino acid, a di, tri, or tetrapeptide. The examples of amino acid residues suitable for the peptide chain include, L-alanine, L-valine, L-leucine, L-isoleucine, L-α-aminobutyric acid, L-phenylalanine, L-tryptophane, L-tyrosine, L-proline, L-hydroxyproline, L-serine, L-threonine, L-cysteine, L-methionine, L-lysine, L-ornithine, L-arginine, L-histidine, L-glutamic acid, L-aspartic acid, L-glutamine and L-asparagine and their D-isomers.

The first residue attached to the muramic acid end is preferably a L-amino acid, selected from the examples mentioned above in the preceding paragraph. L-alanine and L-threonine are preferred.

The second amino acid residue from the muramic acid end, if present as part of a dipeptide, is preferably a D-amino acid. It is preferably D-isoglutamine or an acidic amino acid such as D-glutamic or D-aspartic acid. The carboxylic acid groups may be converted to a mono-, or di-C₁-C₂₂ (preferably C₁-C₆) alkyl ester, mono- or diamide or C₁-C₄ alkyl amide thereof or mixed ester-amide, where one carboxyl group is amidated and the other esterified.

A third amino acid residue from the muramic acid end, if present as a part of a tripeptide is preferably a L-amino acid and L-alanine and L-lysine are preferred.

The fourth amino acid residue from the muramic acid end, if present as a part of a tetrapeptide may be either a L- or a D-amino acid, selected from the examples mentioned above.

The preferred value for n is 1.

Compounds of general formula I are disclosed in U.S. Pat, Nos. 4,395,399 and 6,281,191, US Patent Application Publication No. 2001/0034325 A1 and PCT International Application WO 96/01645 and the preferences set out in these document are equally preferred in the present invention.

One of the compounds for use in the present invention falls within general formula I, where R is a single amino acid (L-Alanine) and is N-acetyl-D-glucosaminyl-(β1-4)-N-acetylmuramyl-L-alanine, the structure of which is:

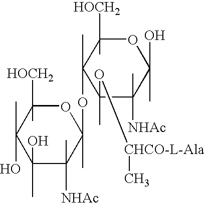

One of the most preferred compounds for use in the present invention falls within general formula I, where the peptide R is (L-Ala-D-isoGln) and is N-acetyl-D-glucosaminyl-(β1-4)-N-acetylmuramyl-L-alanylD-isoglutamine (GMDP), the structure of which is:

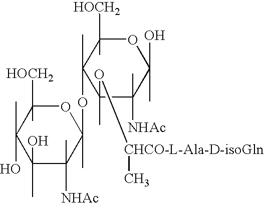

This compound (Compound II in U.S. Pat. No. 4,395,399), also known as glycopin, has already undergone preclinical toxicity testing and pharmacokinetic investigations required for licensing for clinical use in the former USSR.

The acute toxicity in mice, measured by the LD₅₀ test is 7 g/kg. This figure shows the compound to be almost an order of magnitude less toxic than muroctasin, which has an LD₅₀ value in mice of 625 mg/kg. While the pyrogenicity of GMDP is sufficiently low to make it suitable for use in the present invention, and not to have prevented its clinical evaluation for other purposes, it may in some circumstances be preferable to use an even less pyrogenic analogue.

Such an analogue is represented in another preferred embodiment, where R may represent L-Ala-D-Glu and is N-acetyl-D-glucosaminyl-(β1-4)-N-acetylmuramyl-L-alanylD-glutamic acid (GMDP-A), which is Compound III in U.S. Pat. No. 4,395,399, and whose structure is as follows:

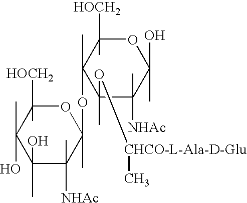

Other preferred compounds within the scope of general formula I include:

N-acetyl-D-glucosaminyl-(βI,4)-N acetylmuramyl-L-alanyl-L-isoglutamine (GMDP-LL) which has the structure:

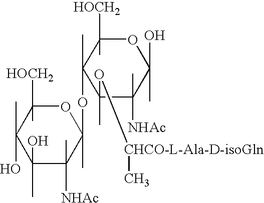

N-acetyl-D-glucosaminyl-(βI,4)-N-acetylmuramyl-L-alanyl-D-glutamine n-butyl ester (GMDP-OBu) which has the structure:

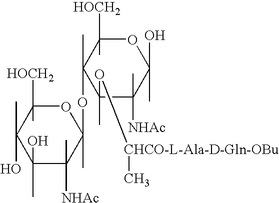

N-acetyl-D-glucosaminyl-(βI,4)-N-acetylmuramyl-L-alanyl-D-isoglutaminyl-L-lysine (GMDP-Lys) which has the structure:

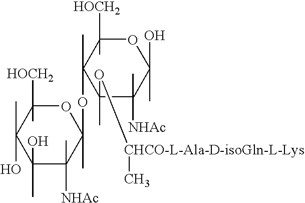

N^{α}-[N-acetyl-D-giucosaminyl-(βI,4)-N-acetylmuramyl-L-alanyl-D-isoglutaminyl]-N^{ε}-stearoyl-L-lysine (GMDP-Lys (St)) which has the structure:

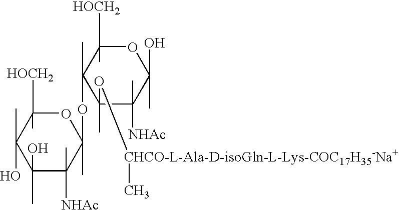

N^{α}-[N-acetyl-D-glucosaminyl-(βI,4)-N-acetylmuramyl-L-alanyI-γ-D-glutaminyl]-N^{ε}-stearoyI-L-lysine (GMDPA-Lys (St)) which has the structure:

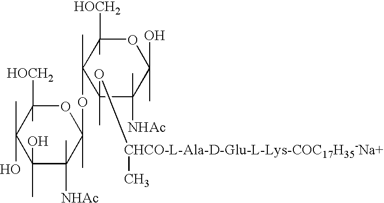

N-acetyl-D-glucosaminyl-(βI,4)-N-acetylmuramyl-N-methyl-L-alanyl-D-isoglutamine, (Me-GMDP) which has the structure

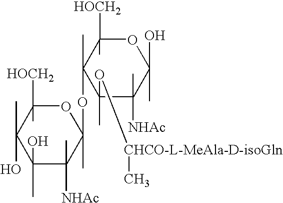

L-Threonyl-N^{ε}-[N-acetyl-D-glucosaminyl-(βI,4)-N-acetylmuramyl-L-alanyl-γ-D-isoglutaminyl]-L-lysyl-L-prolyl-L-arginine (GMDP-tuftsin E) which has the structure:

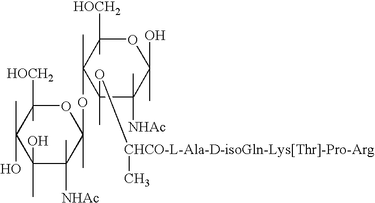

N-acetyl-D-glucosaminyl-(PI3,4)-N-acetylmuramyl-L-alanyi-γ-D-isoglutaminyl-L-threonyl-L-lysyl-L-prolyl-L-arginine (GMDP-tuftsin A) which has the structure:

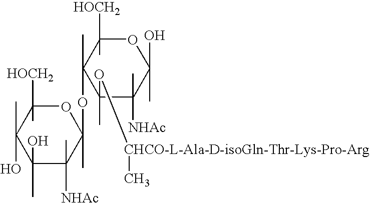

N-acetyl-D-glucosaminyl-(βI,4)-N-acetylmuramyl-L-alanyl-D-isoglutaminyl-L-glutamyl-L-tryptophan (GMDP-thymogen I) which has the structure:

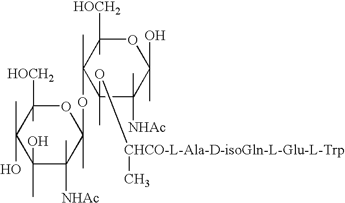

N-acetyl-D-glucosaminyl-(βI,4)-N-acetyimuramyl-L-alanyl-D-isoglutaminyl-E-aminohexanoyl-L-glutamyl-L-tryptophan (GMDP-thymogen II) which has the structure:

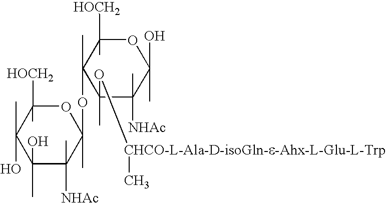

N^{α}-[N-acetyl-D-glucosaminyl-(βI,4)-N-acetylmuramyl-L-alanyl-D-glutaminyl]-N^{ε}-stearoyl-L-lysyl-L-glutamyl-L-tryptophan (GMDP-thymogen III) which has the structure:

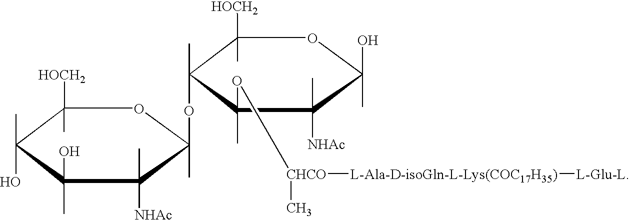

The most preferred compounds are GMDP and GMDP-A.

Glucosaminyl-muramyl dipeptides within the scope of general formula I can be prepared relatively cheaply and in reasonably large quantities by the process disclosed in U.S. Pat. No. 4,395,399. The preparation disclosed is based on the extraction and purification of the disaccharide component from the bacterium Micrococcus lysodecticus and its subsequent chemical linkage to a dipeptide synthesised for example by conventional peptide chemistry. However, the disaccharide may equally well be chemically synthesised using standard sugar chemistry (See for example, Ledvina et al, 1998. Coll. Czech. Chem. Comm. 63, 577-589 and references cited therein; Kiso et al, 1982. Carbohydrate Res. 104, 253-269; Blaszczak et al, PCT Int. Appl. 2001 WO 0179267 and WO 0179268; Vosika & Ma, PCT Int. Appl. 1997 WO 9712894 and Bezonikova, PCT Int. Appl. 1997 WO 9710259).

The present inventors have discovered a novel composition and method for the delivery of a glycopeptide to a patient in need of such treatment, comprising the intranasal administration of a glycopeptide. This composition and method offers significant clinical advantages over the prior art. More specifically, the inventors sought to provide a safe, effective, fast and convenient treatment for administering a glycopeptide to a patient in need of such treatment, which comprises mucosal, particularly intranasal, administration of the glycopeptide intranasally, thus avoiding the side-effects and other disadvantages associated with oral dosage forms. Specifically, smaller doses of a glycopeptide, such as GMDP or GMDP-A, can be administered through the nasal route, thus resulting in rapid onset of action, fewer side effects and reducing drug—drug interactions. By using the composition and method of the present invention, the drug will become more tolerable and more effective in treating patients suffering from ALS or other metabolic and autoimmune disorders.

The pharmaceutical formulations of the present invention comprise of at least one active ingredient, for example, a glycopeptide and optionally other therapeutic agents. The above method may be practiced by administration of the compounds by themselves or In a combination with other active ingredients in a pharmaceutical composition. Other therapeutic agents suitable for use herein are any compatible drugs that are effective by the same or other mechanisms for the intended purpose, or drugs that are complementary to those of the present agents, e.g., antioxidant flavones or any other agents used for disorders described above. The compounds utilized in combination therapy may be administered simultaneously, in either separate or combined formulations, by different routes of administrations or at different times than the present compounds, e.g., sequentially, such that a combined effect is achieved. The amounts and regime of administration will be adjusted by the practitioner, by preferably initially lowering their standard doses and titrating the results obtained. The therapeutic method of the invention may be used in conjunction with other therapies as determined by the practitioner.

Mucosal, preferably intranasal, administration of glycopeptide is more effective than oral administration, and may be conveniently and painlessly self-administered by the patient, and at lower doses and faster onset of action compared to oral dosage forms, thereby allowing a decreased incidence of side effects and decreased incidence of drug—drug interactions and faster onset of action compared to the oral administration.

The nasal route of administration emerged as an efficient local delivery route for OTC drugs many years ago and is an attractive alternative to the parenteral and oral routes. OTC nasal dosage forms mostly use a squeeze bottle or a dropper, which delivers approximate dose. In contrast, Rx nasal medications are delivered accurately with metering devices, which may be either multidose or unit-dose, producing a spray of expelled formulation directed into the nasal cavity. The active ingredient is absorbed through the nasal mucosa and reaches the systemic circulation via numerous capillary vessels present underneath the mucosa. Besides the significant advantages over parenteral and oral routes, the nasal devices eliminate the pain and the fear associated with the needle, thus enhancing patient compliance. Multidose Metering Spray Pumps are specially suited for repeated administrations for treatment of chronic diseases disclosed by this invention and manufacturing of these devices is well known in the art (Devillers 2003. Drug Deliv. Tech. 3, 38).

Moreover, the advantage of the present invention is to provide an improved method for delivery of the glycopeptides and flavone derivatives to increase their bioavailability. The composition permits administration of glycopeptides and flavonoids through the mucosal membrane. The preferred mucosal membranes are nasal, rectal, vaginal, dermal, buccal and sublingual. The most preferred mode of administration for glycopeptides is the nasal route, whereas the most preferred mode of administration for flavonoids is the buccal/sublingual route as previously described in our co-pending U.S. Provisional Patent Application No. 60/407,125 entitled “Parenteral Administration of 3-Dexoyflavinoids to Avoid First Pass Metabolism” filed Aug. 30, 2002, referred to an incorporated hereabove.

Still further, an advantage of the present invention is to provide a method of delivering nutraceutical and therapeutic agents to an individual that provides for increased absorption and bioavailability as compared to medicaments that are designed to be absorbed in the GI tract.

Further, an advantage of the present invention is to provide a method of administering a nutraceutical and therapeutic agents to an individual at a lower level than is typically administered orally while still achieving the same effect.

Furthermore, an advantage of the present invention is to provide a method for administering nutraceutical and therapeutic agents to an individual that heretofore were administered orally.

Moreover, an advantage of the present invention is to provide an improved method for delivery. The composition permits administration of glycopeptides, particularly GMDP and GMDP-A, through nasal, bucket or sublingual mucosa, for attaining sustained blood levels of the active agent.

Still further, an advantage of the present Invention is to provide a method that permits simultaneous, separate or sequential administration of flavonoids, particularly luteolin or its derivatives, through the membranes of the mouth, buccally or sublingually, for attaining sustained blood levels of this active agent.

A method of providing therapy using the pharmaceutical composition of the present invention comprises the application of a dosage form according to this invention to the nasal mucosa, buccal pouch or under the tongue of a subject, such as a human.

Accordingly, a major object of the present invention is to provide a composition and method for the safe, convenient and effective way of administering the glycopeptides to a patient in need of such treatment. The method comprises intranasal administration of an effective amount of a glycopeptide, for example, GMDP or GMDP-A, for the treatment of ALS or other metabolic and autoimmune disorders.

In addition, nasal GMDP administration is easy and convenient in ALS patients, where swallowing of oral dosage forms is painful and difficult. Furthermore, in many situations it has already been shown that the onset and extent of drug delivery after intranasal administration is comparable to the same drug and dose being given intravenously. Therefore, intranasal delivery of GMDP for treatment of ALS or other metabolic and autoimmune disorders could be used in those situations where a rapid or intermittent drug effect is desired.

In certain embodiments, the invention Is directed to a method of providing glycopeptide therapy to a patient in need thereof comprising intranasally administering an effective amount of a glycopeptide or a pharmaceutically acceptable derivative thereof to said patient and compositions thereof. Preferably, the glycopepuide is administered with a pharmaceutically acceptable carrier which can be in the form of, e.g. a solution, suspension, gel, ointment, lotion, semi-solid, vaporizable carrier, a powder and combination thereof. In certain embodiments, the carrier can provide a sustained release of the drug.

A still further aspect of this invention is a pharmaceutical composition of matter that comprises a glycopeptide as described above, and/or pharmaceutically acceptable derivative thereof, and at least one pharmaceutically acceptable carrier suitable for nasal administration.

Suitable carriers are well known to those skilled in the art and vary with the desired form and mode of administration of the pharmaceutical composition. Typically, the carrier must be biologically acceptable and inert and may be a liquid, solution, suspension, gel, ointment, lotion, semi-solid, or vaporizable carrier, or combinations thereof. In a preferred embodiment, the carrier is a pharmaceutically acceptable aqueous carrier. Such compositions are prepared in accordance with accepted pharmaceutical procedures, for example, as described in Remington's Pharmaceutical Sciences, seventeen edition, ed. Alfonso R. Gennaro, Mack Publishing Company, Easton, Pa., Eighteenth edition (1990), which is hereby incorporated by reference. The drug can also be in powder form without the need for further excipient.

The glycopeptide compounds of the invention may be formulated together with the carrier into any desired multidose or unit dosage form. Unit dosage forms such as solutions, suspensions, and water-miscible semisolids are particularly preferred. To prepare formulations suitable for intranasal administration, solutions and suspensions are sterilized and are preferably isotonic to blood.

The formulations may conveniently be presented in unit dosage form and may be prepared by any method known in the art. Such methods include the step of bringing the active ingredient into association with the carrier which itself may encompass one or more accessory Ingredients. In general, the formulations are prepared by uniformly and intimately bringing into association the active ingredient with liquid carriers or finely divided solid carriers or both, and then if necessary shaping the product. Various unit dose and multidose containers, e.g., sealed ampules and vials, may be used, as is well known in the art (see Remington's Pharmaceutical Sciences, seventeenth edition, ed. Alfonso R. Gennaro, Mack Publishing Company, Easton, Pa., Eighteenth edition, 1990). For example, the glycopeptide can be administered into the nasal passages by means of a simple dropper a dispensing tube from which the contents are expelled drop by drop by means of air pressure provided by a manually powered pump, e.g., a flexible rubber bulb, attached to one end. Fine droplets and sprays can be provided by a manual or electrically powered intranasal pump dispenser or squeeze bottle as well known to the art, e.g., that is designed to blow a mixture of air and fine droplets into the nasal passages. To achieve good plasma concentrations, the glycopeptide may be administered, for instance, by intranasal administration of an approximate 0.1 to 1M solution of the active ingredient, optionally in saline.

In addition to the ingredients particularly mentioned above, the formulations of this invention may also include other agents conventional in the art for this type of pharmaceutical formulation. For example, the nasal formulation can provide a sustained release of the drug in order for e.g., once or twice daily. Suitable sustained release materials include cellulose derivatives which adheres to the nasal mucosa, as described in EP 205282, hereby incorporated by reference. The use of the bioadhesive microspheres in drug delivery compositions for transmucosal administration has been described in WO 88/09163 and WO 89/03207, which are hereby incorporated by reference.

The ion-exchange microspheres, described in U.S. Pat. No. 5,935,604 and hereby Incorporated by reference, can also be suitably used in the present invention, where the glycopeptide is bound to the microsphere, which carry suitable anionic groups such as carboxyl groups, carboxymethyl groups, sulphopropyl groups and methylsulphonate groups. Here the strong binding of the drug to the microspheres via a process of ionic interaction has been used to modify drug release rates. Carboxylated starch microspheres are especially preferred. Other materials include hyaluronic acid, chondroitin sulphate, alginate etc. as described in Kwon, et al., J. Colloid Interface Sci. 143, 501. Cationic ion-exchange resins containing sulphonate, carboxyl, carboxymethyl or sulphopropyl groups can also be used and these Include, for example, carboxymethyl dextran (CM Sephadex™) and sulphopropyl dextran (SP Sephadex™.) carboxymethyl agarose (CM, Sepharose™.), carboxymethyl cellulose, cellulose phosphate, sulphoxyethyl cellulose, agarose and the like available from Pharmacia. Cation exchangers such as Amberlite or Dowex containing strongly acidic sulphonic acid group or weakly acidic carboxylic acid group are also useful. In a liquid formulation, the polymeric ion-exchange material will typically be provided in a concentration of from 0.01% to 20%, preferably 0.05-10%, more preferably 0.1%-5%.

The composition may also be a liquid formulation comprising a polymeric ion-exchange material. The polymeric material should provide a negatively charged group as discussed above and also should provide a viscous solution to aid retention in the nasal cavity. Preferably the material will gel when in contact with the nasal mucosa.

Suitable polymeric materials include gellan gum, welan, rhamsan, alginate, carboxymethylcellulose, sodium alginate, xanthan, agar, guar derivatives such as carboxymethyl guar gum, carageenan, dextran sulphate, keratan, dermatan, pectin. Polysaccharides and derivatives are particularly suitable (“Polysaccharides and derviatives” edited by R C Whistler and J N BeMiller (3rd Ed.) Academic Press, San Diego 1993). A preferred material is gellan gum (Gelrite™ from Kelco), which is the deacetylated form of the extracellular polysaccharide from Pseudomonas elodae. The advantage of gellan over other materials is that when administered as a fluid system in the nasal cavity the system undergoes a phase transition forming a gel, thereby providing a bioadhesive effect, holding and releasing the drug at the mucosal surface for an extended period of time. The gellan can be prepared at a concentration of 0.1 w/v to 15% but a preferred range of concentrations is 0.2% to 1%.

Another preferred polysaccharide material is alginate. Mixtures of gellan with other polymers such as alginate can be used, gelling of the mixture being caused by the gellan gum. Other combinations of gums can also be used, particularly where the combination gives a synergistic effect, for example in terms of gelation properties. An example is xanthan-locust bean gum combinations.

For gelling to occur, particularly of gellan gum, monovalent or divalent cations must be present in the composition. Suitable cations include sodium, potassium, magnesium and calcium. The ionic concentration is chosen according to the degree of gelling required, and allowing for the effect that the ionized drug present may have on gelling. A finite concentration of each cation is required to induce gelation. For the nasal formulations, the ionic strength is kept sufficiently low to obtain a low viscosity formulation but sufficiently high to ensure gelation once administration into the nasal cavity where gelation will take place due to the presence of cations in the nasal liquid.

The liquid formulations are administered using well-lknown nasal spray devices. If the formulations are freeze-dried, they can be reconstituted prior to administration. The liquid formulations of the glycopeptides are preferably preserved at around 5° C. to prevent any breakdown of the compound.

The glycopeptides of this invention may also be administered as an aerosol formulation to the lower respiratory tract. The aerosol formulation comprises a hydrofluoroalkane (HFA) propellant, a pharmaceutically active glycopeptide dispersible in the propellant; and a surfactant, selected from a C₈-C₁₈ fatty acid or salt thereof, a bile salt, a phospholipid, or an alkyl saccharide, which enhances the systemic absorption of the glycopeptide as described in U.S. Pat. No. 6,524,557, which is incorporated herein by reference in its entirety.

“Propellants” used herein mean pharmacologically inert liquids with boiling points from about room temperature (25° C.) to about −25° C., which singly or in combination exert a high vapor pressure at room temperature. Upon activation of the Metered Dose Inhaler (MDI) system, the high vapor pressure of the propellant in the device forces a metered amount of drug formulation out through the metering valve. Then the propellant very rapidly vaporizes dispersing the drug particles. The propellant may comprise one or more of 1,1,1,2-tetrafluoroethane (P134a), 1,1,1,2,3,3,3-heptafluoropropane (P227) and 1,1-difluoroethane (P152a), for example, optionally in admixture with one or more other propellants. Preferably the propellant comprises P134a or P227, or a mixture of P134a and P227, for example a density-matched mixture of p134a and P227.

The surfactants employed in the present invention are surprisingly suitable for use with HFA propellants; their capabilities for enhancement of the absorption of polypeptide give them a dual function, which makes them especially beneficial for use in the present glycopeptide aerosol formulations.

Of the fatty acids and salts thereof, C₈-C₁₈ fatty acids salts are preferred. Examples of preferred fatty acid salts are sodium, potassium and lysine salts of caprylate (C₈), caprate (C₁₀), laurate (C₁₂), myristate (C₁₄) and oleate (C₈). As the nature of the counterion is not of special significance, any of the salts of the fatty acids are potentially useful. A particularly preferred fatty acid salt is sodium caprate.

Suitable bile salts may be for example salts of cholic acid, chenodeoxycholic acid, glycocholic acid, taurocholic acid, glycochenodeoxycholic acid, taurochenodeoxycholic acid, deoxycholic acid, glycodeoxycholic acid, taurodeoxycholic acid, lithocholic acid, and ursodeoxycholic acid.

Of the bile salts, trihydroxy bile salts are preferred. More preferred are the salts of cholic, glycocholic and taurocholic acids, especially the sodium and potassium salts thereof. The most preferred bile salt is sodium taurocholate.

Suitable phospholipids may be for example single-chain phospholipids, for example lysophosphatidylcholine, lysophosphatidylglycerol, lysophosphatidylethanolamine, lysophosphatidylinositol and lysophosphatidylserine or double-chain phospholipids, for example diacylphosphatidylcholines, diacylphosphatidylglycerols, diacylphosphatidylethanolamines, diacylphosphatidylinositols and diacylphosphatidylserines.

Of the phospholipids, diacylphosphatidylglycerols and diacylphosphatidylcholines are preferred, for example dioctanoylphosphatidylglycerol and dioctanoylphosphatidylcholine.

Suitable alkyl saccharides may be for example alkyl glucosides or alkyl maltosides, such as decyl glucoside and dodecyl maltoside.

The most preferred surfactants are bile salts.

The invention is further illustrated by the following examples, which should be considered purely as non-limiting examples, since the technology described can be applied without distinction to all active ingredients with good absorption results. It should be understood that various changes and modifications to the presently preferred embodiments described herein will be apparent to those skilled in the art. Such changes and modifications can be made without departing from the spirit and scope of the present invention and without diminishing its intended advantages. It is therefore intended that such changes and modifications be covered by the appended claims.

### EXAMPLE 1

A 39 year-old male with bulbar onset ALS in final stage was dependent on pure oxygen to maintain a 94% pulsox and a feeding tube for nutrition. If the patient was removed from pure oxygen to ambient air, a drop in his pulsox to 82% occurred within 3 minutes and respiratory distress occurred. The patient had lost all ability to voluntarily move any muscle group In his body for over 8 months except his eyelids. Patient was fed 400 mg luteolin with 400 mg rutin four times a day via aqueous suspension into his feeding tube and administered 6 mg GMDP as an aqueous oral spray. Within 24 hours of GMDP administration, the patient's pulsox rose to 99%-100% with markedly improved pallor. Upon removal of the oxygen 14 hours after the first GMDP dose, patient was able to maintain a 99% pulsox without any respiratory distress. On day two, an additional 6 mg of GMDP was administered as an aqueous oral spray and the patient's small fingers began to respond to voluntary challenge within 2 hours of administration. On day three following the administration of aqueous oral spray containing 6 mg of GMDP, the patient was able to move all fingers on both hands excluding his thumbs as well as slight voluntary movement of both legs at the knee while inclined in his wheelchair. The patient was also able to smile for the first time in over 6 months. After the fourth day of 6 mg of aqueous oral administration of GMDP, the patient began to speak soft-spoken words for the first time in 18 months. The patient's wife was able to clearly understand the patient's sentence. Continued voluntary motor improvement was observed in all four limbs progressively for 3 months with semi-weekly administration of the aqueous oral spray of GMDP with increasing upper and lower limb movement and ease in swallowing of soft foods and viscous liquids. The grip of the patient increased progressively to allow the patient to grasp the hand of his wife and pull her toward him gently using thumbs, albeit weakly.

### EXAMPLE 2

A 50 year-old Male with rapidly progressing bulbar onset ALS experiencing upper and lower limb weakness, ataxia, dysphagia, and moderate vocal impairment had been taking 2000 mg luteolin and 2000 mg rutin per day for 7 months was given 10 mg GMDP as an aqueous oral spray. The patient's voice and energy dramatically improved within 40 minutes peaking in improvement at one hour. Intermittent intranasal use of 0.291-1.25 mg of GMDP for one month and subsequent use of GMDP-A for four months as an aqueous intranasal spray 0.9-1.25 mg every 36 to 48 hours dramatically reduced fasciculations, improved the patient's mood, energy, physical strength, swallowing ability, and clarity voice. Patient remains stable after 5 months with no progression in his ALS.

### EXAMPLE 3

A 50 year-old male diagnosed with lower limb onset ALS on Sep. 03, 02 had symptoms of brisk reflexes, moderate muscle atrophy, weakness, fatigue, and fasciculations in both legs including foot drop in the right leg. Due to the patient's weakness and instability in his legs, he was prescribed an AFO brace for his right leg in December of 2001. EMG results showed active denervation in both lower extremities, being quite extensive in the right lower extremity. The patient was unable to walk distances in excess of 15 meters with his AFO. Following his diagnosis with ALS, the patient followed a diet of low carbohydrate intake and high protein intake and began taking 1200-1800 mg of luteolin and 1200-1800 mg of rutin daily. His fasciculations decreased dramatically and his disease slowed in progress. In February of 2003, the patient began therapy with GMDP. He initially took 1.51 mg of GMDP intranasally per day for one week with substantial improvement in his leg strength and fasciculations noted within 24 hours. After 2 weeks of daily use of the GMDP, the patient could walk without AFO for distances of up to 200 meters, had periods of no fasciculations, and experienced a peacefulness of mind and body he had not felt in over 2 years.

The patient then used GMDP at varying doses of 0.5 to 1.5 mg per day intranasally every 36 to 96 hours and it was found that 1.42 mg every 96 hours was the optimal frequency and dose.

The patient then tried to take GMDP more frequently than every 96 hours and he experienced significant joint and muscle pain in his legs and arms. This side affect was attributed to GMDP stimulation of TNF-α lpha synthesis that bound to the p75 receptor or TNFR-2 causing temporary (acute) sinovitis. This is the currently accepted mechanism of rheumatoid arthritis. The patient then took additional luteolin and rutin increasing his dose to 2000 mg of each per day to block excess TNF-α lpha synthesis and he reported a dramatic reduction in his sinovitis.

The patient then took a drug holiday for a period of 10 days and restarted his ALS therapy with GMDP-A. The patient took 0.31 mg as an aqueous intranasal spray daily for three to four days with 24-96 hours off between every three to four day cycle. After observing little or no side affects observed with the 0.31 mg dose, the patient increased his dose to 0.93 mg GMDP-A every 96 hours. The patient reported major reductions in his fasciculations as observed prior with GMDP but with no side affects and he felt as if ALS had left his body. After 4 months of therapy with GMDP-A, the patient has found that 1.42 mg of GMDP-A every 96 hours has abated all progression and symptoms of ALS and he is currently rehabilitating his muscles with light physical therapy on a daily basis.

### EXAMPLE 4

A 51 year-old male diagnosed with upper limb onset ALS observed a reduction in his symptom progression over the past two years while taking 1000 mg of luteolin and 1000 mg of rutin daily. However, the patient had recently lost the ability to use both of his arms, including dressing, combing his hair, or feeding himself. The patient was given 0.942 mg GMDPA as an intranasal spray on a daily basis. Patient observed a 90% reduction in fasciculations within 2 days, and his ability to comb his hair and feed himself was restored, concurrent with dramatic mood improvement. No progression of his ALS was observed for 4 months.

### EXAMPLE 5

A 57 year-old female ALS patient diagnosed 8 years prior with multiple limb and bulbar ALS observed symptom reductions and stopping of disease progression over the past two years while taking 1200 mg of luteolin and 1200 mg of rutin daily. Patient remained confined to wheelchair but was able to stand for increasing periods of up to 142 seconds while taking the luteolin/rutin combination. She started GMDP-A therapy as an intranasal spray for 3 months with the following dose and frequency: Day one 1.24 mg, day two 0.93 mg, day three 0.62 mg, day four 0.31 mg, and day five 0.31 mg. Day 6 was a drug holiday and the cycle was repeated every 6 days. Patient reported a 100% reduction in fasciculations, no progression of disease, and ability to stand with support and walk with a walker for short periods of time.

### EXAMPLE 6

A 45 year-old female ALS patient diagnosed 3 years prior with multiple limb and bulbar ALS observed symptom reductions and slowing of disease progression over the past four months while taking 1800 mg of luteolin and 1800 mg of rutin daily. Patient walked periodically for short distances but could not do so without great fatigue and assistance. Patient could not eat solid foods and required suction to remove aspirated foods and liquids at every meal and was recommended by her physician to get a feeding tube. The patient started GMDP-A therapy as an intranasal spray for 3 months with the following dose and frequency: 1.42 mg ever 96 hours. Patient reported a cessation of her ALS symptoms and increasing endurance, mood elevation, range of motion and strength in her legs and arms, and is now rehabilitating with aquatic physical therapy twice a day and is enjoying daily two-kilometer walks in the forest with her husband or daughter at her side.

### EXAMPLE 7

Twenty five ALS patients were given an aqueous intranasal spray of 0.31 mg of GMDP-A for a period of 1-4 months with the following results: Twenty one patients reported dramatic reductions in their symptoms of fasciculations, muscle weakness, fatigue, and choking with improved strength, swallowing ability, mood, and range of motion within 24 to 96 hours and a desire to maintain the GMDP-A therapy.

1. A method for treating a metabolic or autoimmune disorder in a human or veterinary patient, said method comprising the steps of
(A) administering to the patient a therapeutically effective amount of a compound having the formula: 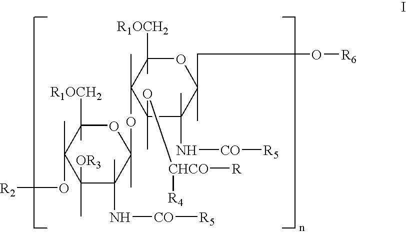
wherein:
R₁, R₂ and R₃ each represents a hydrogen atom or a C₁-C₂₂ acyl group;
R₄ represents a hydrogen atom or a C₁-C₆ alkyl group;
R₅ represents a C₁-C₂, alkyl group or a C₆ or C,₀ aryl group;
R₆ represents a hydrogen atom; and
R represents the residue of an amino acid or a linear peptide of up to from 2 to 6 amino acid residues. Furthermore, at least one of the residues may be optionally substituted with a lipophilic group through an ester or amide bond; and n is 1 and 2; and
(B) administering to the patient a natural or synthetic compound that comprises a flavone, flavonoid, isoflavone or a derivative, prodrug or congener thereof.

2. A method according to claim 1 wherein Step A comprises administering GMDP.

3. A method according to claim 1 wherein Step A comprises administering GMDP-A.

4. A method according to claim 1 wherein Step A comprises administering GMDP and GMDP-A.

5. A method according to claim 4 wherein Step A comprises administering GMDP and GMDP-A in separate doses at separate times.

6. A method according to any of claims 1-5 wherein the compound is administered enterally.

7. A method according to any of claims 1-5 wherein the compound is administered parenterally.

8. A method according to claim 7 wherein the compound is administered intranasally.

9. A method according to claim 7 wherein the compound is administered sublingually.

10. A method according to claim 7 wherein the compound is administered by buccal administration.

11. (canceled)

12. A method according to claim 1 wherein Step A and Step B are carried out substantially simultaneously.

13. A method according to claim 1 wherein Step A and Step B are carried out at different times.

14. A method according to claim 12 wherein the compound of Step A and the compound of Step B are administered in a fixed dosage combination pharmaceutical preparation.

15. (canceled)
